(19) United States
(12) Patent Application Publication
(10) Pub. No.: US 2022/0194905 A1
(43) Pub. Date: Jun. 23, 2022

(54) Method and apparatus for continuous post-treatment of benzotriazole synthetic fluid

(71) Applicant: Jiangsu Yangnong Chemical Group Co., Ltd., Yangzhou, Jiangsu (CN)

(72) Inventors: Genlin WANG, Yangzhou, Jiangsu (CN); Kehong DING, Yangzhou, Jiangsu (CN); Lin XU, Yangzhou, Jiangsu (CN); Gang WANG, Yangzhou, Jiangsu (CN); Yue XU, Yangzhou, Jiangsu (CN); Yuxiu GUO, Yangzhou, Jiangsu (CN)

(21) Appl. No.: 17/606,462

(22) PCT Filed: Apr. 15, 2020
(86) PCT No.: PCT/CN2020/084965

§ 371 Date: Oct. 26, 2021

(30) Foreign Application Priority Data
Apr. 26, 2019 (CN) 201910344876.0

(51) Int. Cl. C07D 249/18; B01J 19/24; B01J 4/00; B01D 11/04; B01D 3/14

(52) U.S. Cl. CPC C07D 249/18; B01J 19/245; B01J 4/001; B01D 2011/002; B01D 3/143; B01J 2219/00033; B01J 2204/002; B01D 11/0492

(57) ABSTRACT

The present invention belongs to the technical field of petrochemical engineering, and relates to a method for continuous post-treatment of benzotriazole (abbreviated as BTA) synthetic fluid. In particular, the present invention relates to a method for synthesizing BTA, including subjecting the BTA synthetic fluid to post-treatment steps of continuous acidification, water washing, extraction, back-extraction, dehydration, and distillation and the like. The method utilizes the difference in solubility of the BTA in water under different pHs to achieve separation by extraction without consuming a large amount of evaporation energy. The present invention is easy to operate, has little environmental pollution, high economic efficiency and low energy consumption, and is easily industrialized.

### CROSS-REFERENCE TO RELATED APPLICATION

The present application is a National Stage of International Patent Application No. PCT/CN20201084965 filed on Apr. 15, 2020, which claims the benefit of priority to Chinese Patent Application Nos. 201910344876.0, filed on Apr. 26, 2019, the disclosures of which are incorporated herein by reference in their entirety.

### TECHNICAL FIELD

The present invention belongs to the technical field of petrochemical engineering, and relates to a method for continuous post-treatment of benzotriazole (abbreviated as BTA) synthetic fluid. In particular, the present invention relates to a method for synthesizing BTA, including subjecting the BTA synthetic fluid to post-treatment steps of continuous acidification, water washing, extraction, back-extraction, dehydration, and distillation and the like.

### BACKGROUND

Benzotriazole (CAS: 95-14-7) is white and light brown needle-like crystals, and is an important fine chemical product. It has a wide range of uses, and is mainly used for an antirust agent and a corrosion inhibitor of metals (silver, copper, lead, nickel, zinc and the like), may also be used as a paint additive, a synthetic detergent preservative, an anticoagulant, a synthetic dye intermediate and the like, with a wide range of downstream applications.

A Chinese patent application (CN105237488A) reports a kettle-type batch synthesis method of BTA, and the BTA is synthesized in one step through a pressure method of o-phenylenediamine. This method all adopts a batch operation, the production efficiency is low, and a recovery method of the BTA in an aqueous layer is not mentioned.

### SUMMARY

The present invention provides a method for continuous post-treatment of BTA synthetic fluid. In particular, the present invention relates to a method for synthesizing BTA, including subjecting the BTA synthetic fluid to post-treatment steps of continuous acidification, water washing, extraction, back-extraction, dehydration, and distillation and the like. The method utilizes the difference in solubility of the BTA in water under different pHs to achieve separation by extraction without consuming a large amount of evaporation energy. The present invention is easy to operate, has little environmental pollution, high economic efficiency and low energy consumption, and is easily industrialized.

A method for continuous post-treatment of the BTA synthetic fluid, including the following steps:

(1) The BTA synthetic fluid and an acidification reagent are continuously fed into an acidification reactor for a continuous acidification, and an acidified aqueous layer and an acidified oil layer are extracted.

(2) The acidified oil layer in (1) is fed into a water washing device for a continuous water washing, and a water-washed oil layer and a water-washed aqueous, layer are extracted.

(3) The, acidified aqueous layer in (1) and the water-washed aqueous layer in (2) are combined, and fed into an extraction tower for a continuous extraction, and an extracted aqueous layer and an extraction oil layer are extracted.

(4) The extracted oil layer in (3) is fed into a back-extraction tower for a continuous back-extraction, and a back-extracted oil layer and a back-extracted aqueous layer are extracted, as to achieve the reuse of an extractant and BTA in the aqueous layer.

(5) The water-washed oil layer in (2) is subjected to a continuous dehydration and a <continuous distillation, as to obtain a BTA product.

In the step (1), the acidification conditions are as follows: an acidification reagent is one or more of a hydrochloric acid, a sulfuric acid, a nitric acid, and an acetic acid, the acidification temperature is 20˜100° C., pH=3˜8, preferably 3˜7 or 3˜6, and further preferably 4˜6 or 3˜5 or 5˜6; and

the residence time is 1 min˜300 min.

Preferably, an acidification temperature is 40˜100° C., 40˜90° C., 40˜80° C., or 40 ˜70° C., or preferably 50˜100° C., 50˜90° C., 50˜80° C., or 50˜70° C., or preferably 60˜100° C., 60˜90° C., 60˜80° C., or 60˜70° C.

Preferably, a pH value of an acidification end point is 3˜6, or the pH value of the acidification end point is 3, 4, 5 or 6.

Preferably, a residence time of acidification is 30 min˜300 min, more preferably 60 min˜300 min, 60 min˜200 min, or 60 min˜100 min, or preferably 70 min˜300 min, 70 min˜200 min, or 70 min˜100 min, or preferably 100 min˜300 min or 100 min˜200 min or 200 min˜300 min.

In the step (2), the water washing conditions are as follows: a temperature of the water washing is 20˜100° C., a ratio of a water washing volume flow rate is water: acidified oil layer=0.1:1˜20:1, and a residence time is 1 min˜300 min.

Preferably, a temperature of the water washing is 40° C.˜90° C., 40° C.˜80° C., 40° C.˜70° C., 40° C.˜60° C., or 40° C.˜0° C.; or preferably 50° C.˜90° C., 50° C.˜80° C., 50° C.˜70° C. or 50° C.˜60° C.; or preferably 60° C.˜90° C., 60° C.˜80° C., or 60° C.˜70° C.; or preferably 70° C.˜90° C., or 70° C.˜80° C.

Preferably, a ratio of the water washing volume flow rate is water: acidified oil layer=0.5:1˜20:1, 1:1˜20:1, 5:1˜20:1 or 1:1˜10:1; or preferably 0.5:1˜10:1, 1:1˜10:1 or 5:1˜10:1.

Preferably, a residence time of water washing is 15 min˜300 min, 30 min˜300 min, 50 min˜300 min, 60 min˜300 min, 100 min˜300 min, or 200 min˜300 min; or preferably 15 min˜250 min, 30 min˜250 min, 50 min˜250 min, 60 min˜250 min, 100 min˜250 min, or 200 min˜250 min; or preferably 15 min˜200 min, 30 min˜200 min, 50 min˜200 min, 60 min˜200 min, or 100 min˜200 min; or preferably 15 min˜100 min, 30 min˜100 min, 50 min˜100 min, or 60 min˜100 min.

In the step (3), the extraction conditions are as follows: a temperature of the extraction is 20˜100° C., a ratio of an extraction volume flow rate is an extractant: (the water-washed aqueous layer+the acidified aqueous layer)=0.1:1˜20:1, and a residence time is 1 min˜300 min.

Preferably, an extraction temperature is 50˜100° C., 50˜90° C., 50˜80° C., or 50˜65° C.; or 60˜100° C., 60˜90° C., or 60˜80° C.; or 65˜100° C., 65˜90° C., or 65˜80° C.; or 80° C.˜90° C.

Preferably, a ratio of the extraction volume flow rate is an extractant: (the water-washed aqueous layer+the acidified aqueous layer)=1:1˜20:1, 1:1˜15:1, 1:1˜10:1, or 1:1˜5:1; or preferably 5:1˜20:1, 5:1˜15:1, or 5:1˜10:1; or preferably 10:1˜20:1, or 10:1˜15:1.

Preferably, a residence time is 5 min˜300 min, 5 min˜200 min, 5 min˜100 min, 5 min˜60 min, or 5 min˜50 min; or 50 min˜300 min, 60 min˜300 min, 100 min˜300 min, or 200 min˜300 min; or preferably 50 min˜200 min, 60 min˜200 min, or 100 min˜200 min.

In the step (3), the extractant is one or more mixtures of chlorobenzene, dichlorobenzene, nitrochlorobenzene, toluene, xylene, benzene, methyl isobutyl ketone (MIBK), dichloromethane, dichloroethane, chloroform, carbon tetrachloride, and ethyl acetate.

In the step (4), conditions of the back-extraction are as follows: a back-extraction temperature is 20˜100° C., a ratio of a back-extraction volume flow rate is a back-extractant: the extracted oil layer=0.1:1˜20:1, a residence time is 1 min˜300 min, and under the better conditions, the residual amount of the BTA in the back-extracted oil layer is less than 0.5%.

Preferably, the back-extraction temperature is 40˜90° C., 50˜90° C., 60˜90° C., or 65˜90° C.; or preferably 40˜80° C., 50˜80° C., 60˜80° C., or 65˜80° C.; or preferably 40˜70° C., 50˜70° C., 60˜70° C., or 65˜70° C.; or preferably 40˜65° C., 50˜65° C., or 60˜65° C.

Preferably, the ratio of the back-extraction volume flow rate is back-extractant: extracted oil layer=0.5:1˜20:1, 1:1˜20:1, 5:1˜20:1, or 10:1˜20:1; or preferably 0.5:1˜10:1, 1:1˜10:1, or 5:1˜10:1; or preferably 0.5:1 ˜15:1, 1:1˜15:1, 5:1˜15: 1, or 10:1˜15:1.

In the step (4), the back-extractant is the same as the acidification reagent in step (1), and is one or more of the hydrochloric acid, the sulfuric acid, the nitric acid, and the acetic acid.

In the step (5), a temperature of a continuous dehydration kettle material is 100˜200° C., preferably 150˜180° C., more preferably 170° C., an absolute pressure is 5˜50 kPa, preferably 5˜30 kPa, 5˜20 kPa, or 5˜10 kPa, more preferably 5 kPa. The continuous distillation absolute pressure is 0˜2 kPa, preferably 1 kPa, and the corresponding gas phase temperature is 140˜190° C., preferably 150˜180° C., or more preferably 180° C.

The present invention further provides a system for continuous post-treatment of the BTA synthetic fluid, and the system is connected to an acidification reactor, a water washing device, a dehydration device, a distillation device, and a molding device in sequence; and the water washing device is also connected to an extractor 1 and an extractor 2 in sequence, and connected to the acidification reactor through the extractor 2.

Further, the acidification reactor is connected with the extractor 1 through an acidified water pipeline.

Further, the extractor 2 is returned to the extractor 1 through a back-extraction oil pipeline.

Further, the dehydration device is returned to the water washing device through a evaporated water pipeline.

A scheme of the present invention is utilized, and a method for continuous post-treatment of the BTA synthetic fluid is provided. The BTA is synthesized by subjecting the BTA synthetic fluid to the post-treatment steps of continuous acidification, water washing, extraction, back-extraction, dehydration, and distillation and the like. The method utilizes the difference in solubility of the BTA in water under different pHs to achieve separation by extraction without consuming a large amount of evaporation energy. The present invention is easy to operate, has little environmental pollution, high economic efficiency and low energy consumption, and is easily industrialized.

### BRIEF DESCRIPTION OF THE DRAWINGS

FIG. 1 is a continuous post-treatment flow diagram, 1 is an acidification reactor, 2 is a water washing device, 3 is a dehydration device, 4 is a distillation device, 5 is a molding device, 6 is an extractor 1, and 7 is an extractor 2.

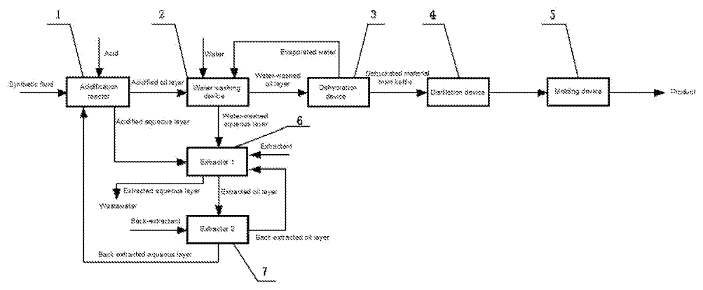

### DETAILED DESCRIPTION OF THE EMBODIMENTS

The following embodiments illustrate the present invention in more detail, but do not further limit the present invention. Unless otherwise specified, herein “%” is “mass %”.

### EXAMPLE 1

BTA synthesis: 295 kg of o-phenylenediamine, 213 kg of sodium nitrite, and 370 kg of water, are put in a 1000 L autoclave, and it is replaced with nitrogen, after stirring evenly, the temperature is raised to 260° C., and hold for 3 h to react. Then the temperature is lowered and the pressure is relieved, and 877 kg of brown clear liquid is released. The analysis shows that the conversion rate of the o-phenylenediamine is 100%, and the yield of the BTA is 98.94%. With this process, the BTA synthesis is performed, and synthetic fluid is subjected to continuous post-treatment.

### EXAMPLE 2

Continuous acidification: the synthetic fluid of Example 1 and 25% hydrochloric acid are continuously fed into an acidification kettle at a weight ratio of about 2:1, the acidification temperature is controlled to 65° C., the residence time is 1 h, and an acidification end point pH=5 ˜6. Acidified liquid is continuously stratified, an obtained acidified oil layer enters a continuous water washing step, and an acidified aqueous layer enters a continuous extraction step.

Continuous water washing: the water washing temperature is controlled to 60° C., the ratio of a feed volume flow rate is water: oil=0.95:1˜1.051, and the residence time is 1 h. An obtained water-washed aqueous layer enters a continuous extraction step, and a water-washed oil layer enters a continuous dehydration step.

Continuous extraction: the water-washed aqueous layer and the acidified aqueous layer are combined, and enter an extraction step. Chlorobenzene is used as an extractant, the extraction temperature is controlled to 60° C., the ratio of the feed volume flow rate is water: oil=1:0.45˜1:0.55, and the residence time is 1 h. An obtained extracted oil layer enters a continuous back-extraction step, and an extracted aqueous layer is treated as wastewater.

Continuous back-extraction: the 25% hydrochloric acid is used as a back-extractant, the temperature is controlled to 65° C., and the residence time is 1 h. The extracted oil layer and the 25% hydrochloric acid are back-extracted according to the ratio of a volume flow rate that is hydrochloric acid: extracted oil layer=0.2:1˜0.25:1, and BTA content in an obtained back-extracted oil layer obtained at this moment is <0.1%. The obtained back-extracted oil layer is used as a recovery extractant and reused to the continuous extraction step, and a back-extracted aqueous layer is BTA hydrochloride, and reused to the continuous acidification step.

Continuous dehydration: the continuous water-washed oil layer is dehydrated, the absolute pressure is controlled to 5 kPa, and the kettle temperature is 170° C., the water content of a kettle material at this moment is less than 0.1%, dehydration water is reused to the continuous water washing step, and the dehydrated kettle material enters a continuous distillation step.

Continuous distillation: the dehydrated kettle material is continuously fed into a distillation tower, the absolute pressure is controlled to 1 kPa, and the temperature of a tower top is about 180° C. A tower top fraction extracted continuously is a BTA product, and the appearance of the product after cooling is a white solid.

### EXAMPLES 3-6

On the basis, of Example 2, the extractant is changed to MIBK, the back-extractant and acid are changed to a sulfuric acid, other conditions are unchanged, and the applications of materials are performed. Results are as follows:

TABLE 1

| Example | Application | Product | Product |
|---|---|---|---|
| number | number | colourity | yield/% |
| Example 3 | First batch | 20 | 81.2 |
| Example 4 | Set 1 | 20 | 97.4 |
| Example 5 | Set 2 | 15 | 97.8 |
| Example 6 | Set 3 | 15 | 98.1 |

### EXAMPLES 7-17

On the basis of Example 2, the post-treatment conditions are changed, and results are as follows:

TABLE 2

| | | | | | Water washing | | | |
|---|---|---|---|---|---|---|---|---|
| | Acidification | | | | | Residence | | Extraction |
| Example | Acid | Temperature | End point | Time | Temperature | time | Water-oil | Extractant |
| No [symbol] | name | [symbol] ° c. | pH | [symbol] min | [symbol] ° c. | min | ratio | name |
| 7 | 35% | 60 | 4 | 60 | 60 | 15 | 0.5 | Chlorobenzene |
| | hydrochloric | | | | | | | |
| 8 | 20% | [symbol] 0 | 3 | 200 | 50 | 30 | 1 | Xylene |
| | hydrochloric | | | | | | | |
| 9 | 40% | 40 | [symbol] | [symbol] 0 | 60 | 100 | 1 | Dichlorobenzene |
| | sulfuric | | | | | | | |
| 10 | 80% | 80 | 5 | 100 | 80 | 100 | 10 | Benzene |
| | sulfuric | | | | | | | |
| 11 | [symbol] 2% | 80 | 6 | 200 | 50 | 200 | 10 | Nitrochlorobenzene |
| | nitric | | | | | | | |
| 12 | 20% | 90 | [symbol] | 100 | 90 | 50 | 5 | Nitrochlorobenzene |
| | nitric | | | | | | | |
| 13 | 50% | 100 | 5 | [symbol] 00 | [symbol] 0 | 300 | 20 | Dichlorobenzene |
| | acetic | | | | | | | |
| 14 | 30% | 70 | 6 | 100 | 70 | 100 | 0.5 | Chlorobenzene |
| | acetic | | | | | | | |
| 15 | 25% | 65 | 8 | 60 | 60 | 60 | 10 | Chlorobenzene |
| | hydrochloric | | | | | | | |
| 16 | 25% | 65 | 1 | 60 | 60 | 60 | 10 | Chlorobenzene |
| | hydrochloric | | | | | | | |
| 17 | 25% | 65 | [symbol] | 60 | 60 | 60 | 0.05 | Chlorobenzene |
| | hydrochloric | | | | | | | |

| | Extraction | | | Back-extraction | | |
|---|---|---|---|---|---|---|
| | | | Residence | | | Residence |
| Example | Temperature | Oil-water | time | Temperature | Acid-oil | time |
| No [symbol] | [symbol] ° c. | ratio | [symbol] min | [symbol] ° c. | ratio | [symbol] min |
| 7 | 60 | 20 | 5 | 60 | 0.5 | [symbol] |
| 8 | 50 | 15 | 100 | 30 | 10 | [symbol] 0 |
| 9 | 100 | 15 | 300 | 60 | 5 | 200 |
| 10 | 80 | 5 | 50 | 80 | 20 | [symbol] 0 |
| 11 | 90 | 5 | 200 | 50 | 10 | 1 [symbol] 0 |
| 12 | 90 | 5 | 100 | 90 | 5 | 100 |
| 13 | 65 | 1 | 100 | 40 | 20 | 50 |
| 14 | 50 | 1 | 1 [symbol] 0 | 50 | 5 | 100 |
| 15 | 60 | 1 | 60 | 65 | 0.25 | 60 |
| 16 | 60 | 1 | 60 | 65 | 0.25 | 60 |
| 17 | 60 | 1 | 60 | 65 | 0.25 | 60 |

[symbol]

indicates data missing or illegible when filed

### CONTRAST EXAMPLES 1-4

The material of Example 2 of the present invention is treated by an batch-type post-treatment process in the prior art. The water washing conditions refer to Example 2, the water washing temperature is 60° C., the water-oil ratio is 1:1, and the heat preservation time is 1 h. Treatment results are as follows:

TABLE 3

| | | | Total time | |
|---|---|---|---|---|
| Example | Treatment | Acidification | from acidification | BTA |
| Number | mode | end point pH | to dehydration/h | yield/% |
| Example 2 | Continuous | 5 | 2 | 98.94 |
| Example 15 | Continuous | 8 | 2 | 50.61 |
| Example 16 | Continuous | 1 | 2 | 5.13 |
| Example 17 | Continuous | 5 | 2 | 98.96 |
| Contrast | Batch-type | 5 | 10 | 97.85 |
| Example 1 | | | | |
| Contrast | Batch-type | 5 | 5 | 95.56 |
| Example 2 | | | | |
| Contrast | Batch-type | 8 | 10 | 42.27 |
| Example 3 | | | | |
| Contrast | Batch-type | 1 | 10 | 5.05 |
| Example 4 | | | | |

The performances of products of the above Examples 7-14 and Contrast Examples are detected. The product colourity, product purity and product yield are all measured in accordance with an HG/T 3824-2014 quality standard method, and measurement results of the product colourity, purity and wastewater are as follows:

TABLE 4

| Example | Product | Product | Product | Wastewater |
|---|---|---|---|---|
| number | colourity | purity/% | yield^{/%} | TOC/ppm |
| Example 7 | 15 | 99.95 | 98.16 | 489 |
| Example 8 | 13 | 99.91 | 98.54 | 455 |
| Example 9 | 10 | 99.96 | 97.96 | 468 |
| Example 10 | 12 | 99.89 | 98.33 | 473 |
| Example 11 | 14 | 99.81 | 98.12 | 485 |
| Example 12 | 15 | 99.79 | 98.59 | 476 |
| Example 13 | 12 | 99.92 | 98.15 | 464 |
| Example 14 | 10 | 99.9 | 98.21 | 433 |
| Example 15 | >150 | 98.12 | 50.61 | — |
| Example 16 | >150 | 95.01 | 5.13 | — |
| Example 17 | 22 | 99.86 | 98.96 | 486 |
| Contrast | 25 | 99.79 | 97.85 | 4325 |
| Example 1 | | | | |
| Contrast | 30 | 99.52 | 95.56 | 5230 |
| Example 2 | | | | |
| Contrast | >150 | 97.96 | 42.27 | — |
| Example 3 | | | | |
| Contrast | >150 | 94.89 | 5.05 | — |
| Example 4 | | | | |

It may be seen from Table 3 and Table 4 that the pH of the acidification end point has the greater impact on the product quality and yield, and preferably the pH is 5˜6. The continuous process product quality is stable. Compared with the batch-type process, the product colourity is reduced by 30˜50%, the quality is better, the Total Organic Carbon (TOG) of wastewater is reduced by about 90% year-on-year, and the wastewater treatment pressure is significantly reduced. In addition, the water-oil ratio of the water washing step also has the impact on the colourity of the product Compared with the batch-type, the continuous post-treatment may achieve the desired effect with less water.

It may be seen from the above descriptions that the existing BTA post-treatment in the world all adopts a batch-type treatment mode, and it is mainly the batch-type acidification, then the batch-type water washing, and the recovery of the BTA in water through cooling, and crystallizing. There are many devices and high energy consumption. However, the present invention adopts the continuous post-treatment, the operation process is simple, the process reliability is high, and the efficiency is high; before dehydration, the post-treated materials have the same temperature, the heat exchange is reduced, and the energy consumption is reduced; and through the continuous acidification, dehydration, and rectification, the BTA products with the high quality are continuously produced. Compared with the batch-type post-treatment process, the energy consumption is reduced, there are no frequent temperature rising and falling crystallization process and a large number of solid-liquid separation processes of the batch-type treatment, and TOC in the wastewater obtained from the continuous treatment is less than 500 ppm, and the wastewater treatment pressure is also significantly reduced.

The content of the present invention is not limited to the content of the Examples of the present invention.

In this article, specific examples are used to describe the structure and implementation modes of the present invention. The descriptions of the above Examples are only used to help understand the core idea of the present invention. It should be pointed out that for those of ordinary skill in the art, without departing from the principle of the present invention, a plurality of improvements and modifications may be made to the present invention, and these improvements and modifications also fall within a scope of protection of the claims of the present invention.

What is claimed is:

1. A method for continuous post-treatment of benzotriazole (BTA) synthetic fluid, wherein the method comprises the following steps:
(1) continuously feeding the BTA synthetic fluid and an acidification reagent into an acidification reactor for a continuous acidification, and extracting an acidified aqueous layer and an acidified oil layer;
(2) feeding the acidified oil layer in (1) into a water washing device for continuous water washing, and extracting a water-washed oil layer and a water-washed aqueous layer;
(3) combining the acidified aqueous layer in (1) and the water-washed aqueous layer in (2), and feeding into an extraction tower for a continuous extraction, and extracting an extracted aqueous layer and an extracted oil layer;
(4) feeding the extracted oil layer in (3) into a back-extraction tower for a continuous back-extraction, and extracting a back-extracted oil layer and a back-extracted aqueous layer, as to achieve the reuse of an extractant and the BTA in the aqueous layer; and
(5) subjecting the water-washed oil layer in (2) to a continuous dehydration and a continuous distillation, and forming in a molding device, as to obtain a BTA product.

2. The method for the continuous post-treatment of the BTA synthetic fluid according to claim 1, wherein in the step (1), the acidification reagent is one or more of a hydrochloric acid, a sulfuric acid, a nitric acid, and an acetic acid.

3. The method for the continuous post-treatment of the BTA synthetic fluid according to claim 1, wherein in the step (2), conditions of the continuous water washing are as follows: a temperature of the continuous water washing is 20˜100° C.

4. The method for the continuous post-treatment of the BTA synthetic fluid according to claim 1, wherein in the step (3), the extraction conditions are as follows: a extraction temperature is 20˜100° C.

5. The method for the continuous post-treatment of the BTA synthetic fluid according to claim 1, wherein in the step (3), the extractant is one or more mixtures of chlorobenzene, dichlorobenzene, nitrochlorobenzene, toluene, xylene, benzene, methyl isobutyl ketone (MIBK), dichloromethane, dichloroethane, chloroform, carbon tetrachloride, and ethyl acetate.

6. The method for the continuous post-treatment of the BTA synthetic fluid according to claim 1, wherein in the step (4), conditions of the back-extraction are as follows: a back-extraction temperature is 20˜100° C.;
preferably, a ratio of a back-extraction volume flow rate is a back-extractant: the extracted oil layer=0.1:1˜20:1;
preferably, a residence time of back-extraction is 1 min˜300 min; and
preferably, a residual amount of the BTA in the back-extracted oil layer is less than 0.5%.

7. The method for the continuous post-treatment of the BTA synthetic fluid according to claim 1, wherein in the step (4), the back-extractant is one or more of a hydrochloric acid, a sulfuric acid, a nitric acid, and an acetic acid.

8. The method for the continuous post-treatment of the BTA synthetic fluid according to claim 1, wherein in the step (5), a temperature of the continuous dehydration is 100˜200° C.;
preferably, an absolute pressure is 5˜50 kPa;
preferably, a continuous distillation absolute pressure is 0˜2 kPa; and
preferably, a corresponding gas phase temperature is 140˜190° C.

9. A system for the method for the continuous post-treatment of the BTA synthetic fluid according to claim 1, wherein the system is connected to an acidification reactor (1) a water washing device (2), a dehydration device (3), a distillation device (4), and a molding device (5) in sequence; and wherein the water washing device (2) is also connected to an extractor 1 (6) and an extractor 2 (7) in sequence, and connected to the acidification reactor (1) through the extractor 2 (7).

10. The system according to claim 9, wherein the acidification reactor (1) is connected with the extractor 1 (6) through an acidified water pipeline.

11. The system according to claim 9, wherein the extractor 2 (7) is returned to the extractor 1 (6) through a back-extraction oil pipeline.

12. The system according to claim 9, wherein the dehydration device (3) is returned to the water washing device (2) through an evaporated water pipeline.

13. The method for the continuous post-treatment of the BTA synthetic fluid according to claim 1, wherein a temperature of the continuous acidification is 20˜100° C.

14. The method for the continuous post-treatment of the BTA synthetic fluid according to claim 1, wherein a pH of the continuous acidification is 3˜8, more preferably 3˜6, and further preferably 4˜6 or 3˜5 or 5˜6.

15. The method for the continuous post-treatment of the BTA synthetic fluid according to claim 1, wherein a residence time of the continuous acidification is 1 min˜300 min.

16. The method for the continuous post-treatment of the BTA synthetic fluid according to claim 1, wherein in the step (2), a ratio of a water washing volume flow rate is water: the acidified oil layer=0.1:1˜20:1.

17. The method for the continuous post-treatment of the BTA synthetic fluid according to claim 1, wherein in the step (2), a residence time of water washing is 1 min˜300 min.

18. The method for the continuous post-treatment of the BTA synthetic fluid according to claim 1, wherein the water washing device is a water washing tower or a water washing kettle.

19. The method for the continuous post-treatment of the BTA synthetic fluid according to claim 1, wherein in the step (3), a ratio of an extraction volume flow rate is an extractant: (the water-washed aqueous layer+the acidified aqueous layer)=0.1:1˜20:1.

20. The method for the continuous post-treatment of the BTA synthetic fluid according to claim 1, wherein in the step (3), a residence time of the extraction is 1 min˜300 min.
